SEQUENCE LISTING (8 sequences)

SEQ ID NO: 1 - protein, 1020 aa, Brettanomyces bruxellensis
  misc_feature (347)..(347): Xaa can be any naturally occurring amino acid
  misc_feature (550)..(550): Xaa can be any naturally occurring amino acid
  misc_feature (562)..(562): Xaa can be any naturally occurring amino acid
  misc_feature (600)..(600): Xaa can be any naturally occurring amino acid
  misc_feature (612)..(612): Xaa can be any naturally occurring amino acid
  misc_feature (617)..(617): Xaa can be any naturally occurring amino acid
  misc_feature (695)..(695): Xaa can be any naturally occurring amino acid
  misc_feature (770)..(770): Xaa can be any naturally occurring amino acid
  misc_feature (779)..(779): Xaa can be any naturally occurring amino acid
  misc_feature (889)..(889): Xaa can be any naturally occurring amino acid
  misc_feature (946)..(946): Xaa can be any naturally occurring amino acid
  misc_feature (980)..(980): Xaa can be any naturally occurring amino acid
MRLMPQKKRDKKSLSGVEHVNPDNGGDDSGEGDGNBDNADDDDDDDDVNMTMMNDESYYADGSVEEGRVLNSSVDNDLYLKPGFGSARNLCITLRYLLYEQAVDYFYDEFNNRDSKFHDFELLDEQHDANKDETSAKNLHDLSESSEDNVSNTENDAEDDNYDDDSPKIERGDASPEDDDYDDDDDDDDDDKGDEDNTKDTNNDNSVKVISTGIPVGVNDKGSEEDKSLNDENSKVDDENDVSPPKYIEPGSHGEMILKVPESAILSKPSYPSLPNSFSPADEAIGKVVHPILSTSSAIESSLEKQNELKLIKNFNKIYHGFDDDLQNIVKRRKLEKSNQQLGLESXESNSSKTDGKLDQSVSKLMSLGGAANLSLKNLLGRIEDNRSKLNITDIELKNLIMDVRKNRSKWANYNRIGQEELYEACEKVVTELRGYTEHSTPFLNRVSKREAPNYYEIIKKPMDLNTVMRKLRNFQYRSKKQFVDDLMLIWQNCLTYNTDPKHYLRVDAIAMQKKTQSLIPLIPDIVVRDRAEVEKEAALAAKQDKEEQXSEQETENATRSXRAVGAVGTGSKQTAKKHRKGPTQEDNQEEVESKEKTAXLDSKTEEETTTXSATPXGTDTPSTAHDEKHLPINLTKSVSKVDSMTPATASGASGTPPQNIDEENDELDEALEELRTERNEDDDDNDVIEDLEAXTWKTLTANTRYKLCEARGKMFKGNKIDPNVESLYRDENQMANFMNYLTDNGDLVIHKNRYFDEHDDPYLIEYDVXGGVPPIKFXGVNFEQEENAMMEKLLGKGETLDSLPDSLLKVKIKGSNSVIMDNIKTMQQIRKTCFRINLIRQMQTSQFVHRSQMSAPDIKEINFNDIDPLSKLPTRDSLNGEVAFRSLXKSISAILMATGFEKTSPFCASLMTQLAEEHLGNLAKSMKLHEESNSINKMNIRGKSXTNIKNIVQMSLEENGIDKPDIIYTYYKEYLAKRXKKLKDLKTALEEFLKELLRPGLQDINETQFTDNSDQFYDW

SEQ ID NO: 2 - DNA, 5063 bp, Brettanomyces bruxellensis
cttattattggtacaagtttatatatcataaatacaaacacggattaaagaaataaaagaaataaaaatgaaaaaaaagaaacccatcacacgcctaaaaaaaaggataagycatgactctatcacgcrtcacctatgtcttccgccttaagttttatcagacgaaatatctttatcatcaatatcaccctgtgatgcatccgtgaaaatatctttatcgtcgcctttctcytcatyctcatcgttatcatcttcgccawcttcattgattgtrtcaagtatrccatttgaatctttctkytccgtttcaacgctttccttrtgatcgttatygagtgaatcatcattagcaacttttctaattacagaatmtccagcattttctttcgatacctttccatcggtcacacttgttgtcgcgtccaattggccggaactakatgacttttcggtatttrttgtttcwtgcgmttgcgtcttctycttttcttctgctacccatttgtcttgatcgtcaataaagaamgaagtagcaatcagtttcttctttatctgcgtaatctttccattaggaggaatttttggtctattattcctttgtttaatcggtagatcatcatcctcaataattctcaaatcattcaaatctttatattcaacaaacttttcatttggatctgcagtaccrttrgcaatatgctgctggtgtcttcgtaattgcttggcrtacatagtttcacttttcgtaagtaatcgttcataatagtgcttaaggacrccaatttgttttggaagatctttcttccgaagctttggaaacwtgaactctttgaagtcatcatacttctctttcyttctcttattgttcaaytggttaaactgatacgaaagacgcgagtgyaataaatgcaacggaacactagcwgaaagaatwccaaattctttatccaatcccagttctctgaaaccaaagaaatcatccccaatctcctcsgaaaattcaccagtcataaaactgatcagaattatcagtgaactgygtttcgtttatatcttgtaatccaggtcttaacaattccttcaagaattcttccagagcagtcttgagatctttgagtttcttgytccttttagcaagatattccttgtaatacgtgtatattatatcyggcttatcaatgccattttcctcaagagacatttggacaatatttttgatattcgttgragacttacccctaatattcatcttgttaatggarttggactcttcatgcaatttcatcgattttgcragatttccaagatgctcttccgcaagttgagtcattaaagaggcacaaaawggacttgttttttcaaatccrgtrgccatgagtattgcrgaaatagacttcygtaacgatctgaaggcaacttcaccattcaacgaatcyctggttgggagctttgagagcggatcaatatcattgaaattaatttctttgatrtctggtgccgacatttgcgaccggtgcacaaattgagaggtttgcatctgtctgataagattgattctaaaacatgtctttcgaatctgttgcatggtcttaatattatccataataacactgttggagcccttgatcttcactttcagcaaactgtctggaagagaatctaaagtttcacctttgcccagtagcttttccatcatggcattttcctcctgctcaaaattcacacctytraacttaatgggcggcactccaccyytaacrtcatattctatcaaatasggatcatcrtgytcatcaaagtatctattcttgtgaatcaccaaatcgccgttatccgtaagatagttcatgaaattggccatctgattttcgtctctatagagcgactccacatttggatcaattttgtttcctttaaacatcttacctctrgcctcacataacttatayctrgtrttygcagtcaaagttttccacgtggmagcctctaaatcctcaattacatcattatcgtcatcatcttcattcctttctgttcgaagttcctccaaagcttcgtctagttcatcgttttcttcatcaatattttgaggtggagttccagaagcaccggatgctgttgcaggagtcatggaatcgaccttggatacacttttcgtcaggtttattggtaaatgcttttcatcatgygcagttgatggrgtatctgttcccrctggtgtagcagaaaycgttgtcgtttcttcttcagtctttgaatctaaagyygcygtcttttctttagattctacttcttcctgattgtcttcctgggttggaccttttcgatgctttttggccgtttgcttacttccagttccaacagccccaacagccctacyagatcttgtagcattctctgtttcctgctcagawtcttgttcctccttatcctgttttgcagccaatgcagcttctttttccacttcagctctatctctcaccactatatccggaatcaacggtatcaatgactgygttttyttctgcatagctattgcatcaacacgaaggtaatgctttggatccgtgttataagtcaaacagttttgccaaattaacatcaaatcatcyacaaactgctttttgcttctatattggaaatttctcagctttctcattactgtattcaagtccattggcttctttatgatttcataataattaggagcttcccttttagagaccctgtttagaaatggcgtggaatgctcagtataacctctcaactcagtgacaactttctcrcaggcttcataaagctcttcttgcccaattctattgtagtttgcccattttgatctattctttctaacatccattatcaagttyttgagttctatgtctgtaatattaagcttgctyctgttatcctctattcttcctaataaattttttaaagaaaggttrgcggctcccccwagactcataagcttactmacactttgatcaagtttkccatccgttttacttgaattcgattcayctgactcaagccccaattgctgattactyttctcaagcttccgrcgcttgactatattttgtagatcatcatcaaaaccatgatatattttgttaaagttcttaatcaactttagctcattctgcttctcaagacttgattcaatagcagacgatgtgctgagaattggatgaacgacttttccaattgcttcatctgctggtgaaaacgagttcggaagacttggataacttggcttggataatatcgcactttccggaacttttaatatcatttcaccatgtgaacctggttcaatatatttaggaggagacacatcattttcgtcatctactttcgaattttcatcatttaagcttttatcttcctccgatcctttatcattaactccgactgggattcctgtagaaataaccttaactgaattatcattattagtgtcctttgtattatcctcatcacccttatcatcatcatcatcatcatcatcatcatcatagtcatcatcttctggagaagcatcaccacgttcaatttttggactgtcgtcatcataattatcatcttctgcatcattctcagtattgcttacattgtcttcagaggattctgacaaatcgtgcaaattcttcgcagatgtttcatctttattagcatcatgctgttcatccaacaactcaaaatcatgaaacttgctatctctgttattaaactcgtcataaaaataatcaacagcctgttcataaagaagatatcgcaatgttatgcaaagatttcgtgcrgaaccaaatccaggcttcaagtataaatcattgtcaactgaactgttgagaacacgmccttcttcaacactsccatcagcataatatgattcatcattcatcatagtcatattgacatcatcatcatcatcatcatcatcagcattatcatyattaccatcaccttcaccactatcatcaccaccattatccggatttacatgttcaacaccggacaaggacttcttatcgcgttttttttgaggcatcagtcgcattatcattcaaaaattgaacaaaaagattacagtccattaatgcatcccatgctgcaagcttttgctcatctacttcactctttatagattgatcatcagaggatytcaaaatgaagctaaggaggttaaacaattgcggactaaggtaacaattaaaagaatccttcattgaagagtttgacggtcaaccgtcgcagcaacttcttattatttcctttgaatgcctcgagtttataaccggccattattttggtctgttgaacaaaaaacaattatccaaatacagatctgaaatattttaagttgacaccaaacgaaattaaaaattcaccctttaactgcaacgttcttccagaaaagatacccgggccaaggtagctaatatatgtgaaatatgattactgtcaaggaacactaatggatgaatagtgaaatgtgatyctggaagaactttgtagcctaactataatcatgcagacgtatgagttacttaaaaaaaaaaawataratraataaatcgaatataatataaaaatygasaacaaaactactcacctctrggcacgccgatcgacgtagtcyggkytttattttattttcrgargctattctatacatattttttgctaatcgaggataaatcatgtgcgtataatrtgggcactaggyaaactgtaataaytctaccgttttatatattttctttcaaaagtatakaagtttcctactttcggrcaattmtttgtttmttggatcragagtttaagraaggaataccctrttcaaaagracryagtsatcaatttaaacgaatatattggttagacctaggaaagagagctaggaatagagaaagtcatataaaatgccagattttgtggataagacatttgatgcagtatattcagtgtatcagacagcagtgggtgctctgaatggagcatatgataaggccaaggatgctgggcaaagcattatggaatatacaaaaaatagtgcc

SEQ ID NO: 3 - DNA, 21 bp, Brettanomyces bruxellensis
tcttcctccgatcctttatca

SEQ ID NO: 4 - DNA, 21 bp, Brettanomyces bruxellensis
ttgcacgatttgtcagaatcc

SEQ ID NO: 5 - DNA, 25 bp, Brettanomyces bruxellensis
agaagttgaacggccgcatttgcac

SEQ ID NO: 6 - DNA, 25 bp, Brettanomyces bruxellensis
aggattgttgacactctcgccgagg

SEQ ID NO: 7 - DNA, 263 bp, Brettanomyces bruxellensis
tcttcctccgatcctttatcattaactccgactgggattcctgtagaaataaccttaactgaattatcattattagtgtcctttgtattatcctcatcacccttatcatcatcatcatcatcatcatcatcatcatagtcatcatcttctggagaagcatcaccacgttcaatttttggactgtcgtcatcataattatcatcttctgcatcattctcagtattgcttacattgtcttcagaggattctgacaaatcgtgcaa

SEQ ID NO: 8 - DNA, 39 bp, Brettanomyces bruxellensis
acgacgttgtaaaacgactcttcctccgatcctttatca

(19) United States
(12) Patent Application Publication
(10) Pub. No.: US 2018/0312930 A1
(43) Pub. Date: Nov. 1, 2018

(54) METHOD FOR ANALYZING A SAMPLE TO DETECT THE PRESENCE OF SULPHITE-RESISTANT YEASTS OF THE BRETTANOMYCES BRUXELLENSIS SPECIES AND KIT FOR IMPLEMENTING SAME

(71) Applicants: UNIVERSITE DE BORDEAUX, BORDEAUX (FR); INSTITUT POLYTECHNIQUE DE BORDEAUX, TALENCE CEDEX (FR); ECOLE NATIONALE SUPERIEURE DES SCIENCES AGRONOMIQUES DE BORDEAUX AQUITAINE BORDEAUX SCIENC, GRADIGNAN (FR)

(72) Inventors: WARREN ALBERTIN, PODENSAC (FR); ISABELLE MASNEUF-POMAREDE, TALENCE (FR); EMILIEN PELTIER, PAREMPUYRE (FR)

(73) Assignees: UNIVERSITE DE BORDEAUX, BORDEAUX (FR); INSTITUT POLYTECHNIQUE DE BORDEAUX, TALENCE CEDEX (FR); ECOLE NATIONALE SUPERIEURE DES SCIENCES AGRONOMIQUES DE BORDEAUX AQUITAINE BORDEAUX SCIENC, GRADIGNAN (FR)

(21) Appl. No.: 15/769,855

(22) PCT Filed: Oct. 19, 2016
(86) PCT No.: PCT/FR2016/052701

§ 371 Date: Apr. 20, 2018

(30) Foreign Application Priority Data
Oct. 20, 2015 (FR) 1559975

(51) Int. Cl. C12Q 1/6895

(52) U.S. Cl. CPC C12Q 1/6895

(57) ABSTRACT

A method for analyzing a liquid sample, such as wine, in order to detect the possible presence of sulphite-resistant yeasts of the Brettanomyces bruxellensis species. The method includes detecting whether yeasts of the Brettanomyces bruxellensis species belonging to the genetic group of triploid oenological yeasts are found in the sample. If such yeasts are detected, then the sample is deduced to contain sulphite-resistant yeasts of the Brettanomyces bruxellensis species.

### RELATED APPLICATIONS

This application is a § 371 application from PCT/FR2016/052701 filed Oct. 19, 2016, which claims priority from French Patent Application No. 15 59975 filed Oct. 20, 2015, each of which is incorporated herein by reference in its entirety.

### REFERENCE TO ELECTRONIC SEQUENCE

The contents of the electronic sequence listing (seq.txt; Size: 18.1 kilobytes; and Date of Creation: Apr. 20, 2018) is herein incorporate by reference in its entirety.

### FIELD OF THE INVENTION

This invention relates to a method for analyzing a sample, in particular a wine sample, for the possible presence of sulphite-resistant yeasts of the Brettanomyces bruxellensis species as well as to a kit for implementing such a method. The invention also relates to the use, for analyzing the possible presence, in a sample, of sulphite-resistant yeasts of the Brettanomyces bruxellensis species, of a pair of primers capable of amplifying a nucleotide sequence specific to yeasts belonging to the genetic group of oenological triploid yeasts of the Brettanomyces bruxellensis species.

### BACKGROUND OF THE INVENTION

The yeasts of the Brettanomyces bruxellensis (known as well as Dekkera bruxellensis) species represent one of the major alteration sources of fermented beverages, such as wine, beer, cider, etc., and, more especially, the first cause of wines contamination. These yeasts produce in particular important quantities of volatile phenols that give the wine an unpleasant taste and smell, currently described by the terms “stable smell” or “horse sweat”. This contamination can be the cause of important economic losses, especially when it affects red wines let to age in wood barrels. The alteration caused by Brettanomyces bruxellensis affects indeed almost 25% of red wines and it causes a quasi-systematic rejection of the wines by consumers.

The persons responsible of the making of wines have currently at their disposal several methods for fighting against wine alteration caused by Brettanomyces bruxellensis.

Among these methods, it can mentioned the methods of predicting the presence of these yeasts in the wine by means of software, such as those marketed under the names BRET'LESS® and BRETT SCORING®, that evaluate, for a given wine, the contamination risk depending on the composition of musts and wine and on the oenological practices applied on the location where wine is made; the quantitative and semi-quantitative methods for detecting the presence of these yeasts, especially by spreading this species in a selective medium, polymerase chain reaction, or flow cytometry, in particular the semi-quantitative tool Veriflow® Brett, that allows determination of the levels of population of Brettanomyces bruxellensis in a sample; or also the curative methods having as purpose to eliminate the produced volatile phenols, for example by means of adsorbents such as yeast flakes, activated carbon, etc. However, none of these methods proves to be really satisfactory. For example, the curative methods have generally an organoleptic impact on the treated wines.

Another type of methods currently implemented for the protection against the presence of yeasts of the Brettanomyces bruxellensis species in wine are the methods for fighting against these yeasts. Among them, it can mentioned filtration, chitosan treatments, etc. These methods have however the disadvantages of a complex implementation and the need of costly investments and they often have a negative impact on the organoleptic quality of wines.

The most spread method for fighting against yeasts of the Brettanomyces bruxellensis species, as being the less restrictive to implement, is the method known as sulphiting, that consists in adding sulphur dioxide in the wine in order to take advantage especially of the antioxidant and antimicrobial properties of the sulphur. This method proves however to be inefficient when the Brettanomyces bruxellensis strains present in the wine are resistant to sulphites. It was indeed shown by the prior art, as in particular described in the publication of Curtin et al., 2012, in Letters in Applied Microbiology, 55: 56-61, that, in the case of the Brettanomyces bruxellensis species, the phenotypic characteristic of sulphite-resistance depends on the strain.

The implementation of the sulphiting method proves, in addition, to be risky as the systematic use of sulphur dioxide in high dose favors the emergence of new strains resisting to sulphites. For example, it was reported, in the aforementioned publication of Curtin et al., 2012, that, in Australia, after about ten years of intensive use of sulphiting in order to combat Brettanomyces bruxellensis, 85% of isolates are today sulphite-resistant. Moreover, in the recent years, the purpose of the general agriculture policy is to reduce inputs. In oenology, this results in strong society pressure to reduce the doses of sulphite used, associated to a more and more restrictive legislation.

Thus, currently there is a need for a method that allows the implementation of a reasoned strategy of use of sulphites for wine sulphiting, i.e. a use targeted only to the wines containing Brettanomyces bruxellensis yeasts that are sensitive to sulphites, for which sulphiting is useful.

For proposing such a method, various studies have been carried out by the prior art in order to attempt to establish a relation between the genotype of the yeasts of the Brettanomyces bruxellensis species and their phenotypic characteristics, and more particularly their capacity to resist to sulphites. This is the case, for example, of the study described in the aforementioned publication of Curtin et al., 2012, whose purpose is to identify, on the basis of many isolates of Brettanomyces bruxellensis, a potential relation between the sulphite-resistance phenotype and the genotypic markers of these yeasts.

However up to this day no study has made it possible to identify a relation between the sulphite-resistance phenotypic characteristic and the genotypic characteristic in the case of Brettanomyces bruxellensis.

### OBJECT AND SUMMARY OF THE INVENTION

The purpose of this invention is thus to propose a method which allows to detect reliably, in a sample likely to contain yeasts of the Brettanomyces bruxellensis species, in particular a wine sample, the presence of yeasts resistant to sulphites, and, optionally, the content thereof with respect to the total population of yeasts of the Brettanomyces bruxellensis species that are present, so as to be able to avoid, later on, treating by sulphiting wines about which it would then be known that they contain sulphite-resistant yeasts, and for which such a sulphiting treatment would prove inefficient.

An additional objective of the invention is that this method be simple and fast to implement and furthermore at a low cost and by means of devices currently present in oenological analysis laboratories.

### BRIEF DESCRIPTION OF THE DRAWINGS

The invention will be better understood on reading the following description, given by way of wholly nonlimiting example and made with reference to the FIGURES, in which:

FIG. 1 shows electrophoresis gel obtain in accordance with an exemplary embodiment of the claimed invention.

### DETAILED DESCRIPTION OF THE EMBODIMENTS

While the works of the prior art indicated an interdependence relationship between resistance to sulphites and yeasts genotype, it was discovered unexpectedly, at the origin of this invention, by the present inventors, that the resistance to sulphites of the yeasts of the Brettanomyces bruxellensis species is not related to a particular genotype of these yeasts, but is related to their ploidy, i.e. not to the quality of the genes of these yeasts, but to the number of copies of chromosomes that they present. More specifically, it was discovered by the inventors that among the three genetic groups of the Brettanomyces bruxellensis species described up to now, namely the diploid group, the oenological triploid group and the beer triploid group, the strains of the oenological triploid genetic group yeasts, and them alone, are resistant to sulphites. Thus, the presence in a sample, in particular a sample whose microorganisms composition is representative of that of a wine, of yeasts of the Brettanomyces bruxellensis species belonging to the genetic group of oenological triploid yeasts, indicates a resistance to sulphites of yeasts contained in this sample, while the absence of such yeasts indicates a sensitivity to sulphites of the yeasts of this sample. It is in this latter case that the treatment with sulphites is of interest.

By genetic group of oenological triploid yeasts it is meant in this description, in a manner that is conventional, the group of the triploid yeasts whose substrate is mostly the wine, in particular the red wine, the grapes juice or grapes, or the vine-growing and wine-producing material. By genetic group of beer triploid yeasts it is meant the group of triploid yeasts whose substrate is mostly the beer as well as other fermented drinks, such as cider, kombucha, tequila, etc. In the publication of Albertin et al., 2014, in Food Microbiology, 42: 188-195, are indicated examples of strains belonging to these groups, indicating for each the associated substrate and the collection in which it is preserved and where it is possible to obtain it.

Thus, according to a first aspect, it is proposed, according to this invention, an analysis method for analyzing a sample, in particular a fraction of a liquid substance, such as a fermented drink, especially a wine, or a sample placed on a solid or semi-solid medium, for the possible presence of yeasts of the Brettanomyces bruxellensis species resistant to sulphites. This method comprises:
a step of detection of the possible presence, in the sample, of yeasts of the Brettanomyces bruxellensis species belonging to the genetic group of oenological triploid yeasts, and
if such a presence of yeasts of the Brettanomyces bruxellensis species belonging to the genetic group of oenological triploid yeasts is detected, the deduction that the sample contains sulphite-resistant yeasts of the Brettanomyces bruxellensis species.

On the contrary, if a presence of yeasts of the Brettanomyces bruxellensis species belonging to the genetic group of oenological triploid yeasts is not detected, it is deduced, if the sample contains yeasts of the Brettanomyces bruxellensis species, that these yeasts are sensitive to sulphites.

According to this invention, the phrases “yeasts resistant to sulphites” and “sulphite-resistant yeasts” mean yeasts with the capacity to grow in the presence of a concentration of sulphite(s) corresponding to the practice in oenology, namely 0.4 mg/L of molecular sulphur dioxide (H₂SO₃) in the medium. The sensitivity to sulphites corresponds to the incapacity of yeasts to grow in the presence of a concentration of molecular sulphur dioxide corresponding to a quantity higher or equal to 0.4 mg/L in the medium.

By sulphites it is referred to the molecular active fraction of sulphur dioxide present in a given medium. This active fraction can in particular result from the addition in the medium, especially in the grapes juice or wine, of salts or esters of sulphurous acid, such as sulphurous anhydride, sodium sulphite, sodium bisulphite, sodium metabisulphite, potassium metabisulphite, potassium sulphite, calcium sulphite, calcium bisulphite, potassium bisulphite, etc., as well as sulphur dioxide.

At the end of the implementation of the analysis method according to the invention, the diagnosis result thus obtained allows advantageously to the specialists in the field to adapt their oenological practices for avoiding wine alteration, in particular concerning sulphiting, and especially to limit the use of sulphur dioxide in the wine-making process to the cases where this use proves to be actually useful.

The sample on which the method according to the invention is applied can be a liquid sample, such as a wine fraction. It can also be a sample collected from a surface such as the soil, the wall of a material used for the wine-making process, for example, a tank, etc., and placed on or contained in a solid or semi-solid support. Such solid or semi-solid supports can be boxes, contact blades, optionally agar blades, swabs, towelettes, sponges, etc.

According to particular implementation modes, the method of the invention has, in addition, the following characteristics, implemented separately or in each of their technically operating combinations.

In particular embodiments of the invention, the detection step comprises the detection, for yeasts contained in the sample, in particular in a wine sample, of a molecular marker specific to yeasts of the Brettanomyces bruxellensis species of the genetic group of the oenological triploid yeasts.

Any molecular marker specific to such yeasts can be targeted by the detection step of the method of the invention. It comes within the skills of the person skilled in the art to identify the molecular markers that allow differentiating the yeasts of the Brettanomyces bruxellensis species of the genetic group of oenological triploid yeasts from those of the oenological diploid and beer triploid genetic groups, in particular on the basis of his general knowledge and of the data published concerning these yeasts, especially the genotypic data.

In particular, it was discovered by the inventors that the protein sequence SEQ ID no. 1 (Genbank accession number: EIF47840.1) is specific to the yeasts of the Brettanomyces bruxellensis species of the oenological triploid group. The protein having this sequence, produced in particular by the strain AWRI1499 of Brettanomyces bruxellensis, is known as “putative histone acetyltransferase saga complex component”. The strain AWRI1499 is available in the collection AWMCC (AWRI Wine Microorganism Culture Collection, Glen Osmond, Australia).

Thus, in particular embodiments of the invention, the detection step of the analysis method comprises the detection, for yeasts contained in the sample, of a molecular marker associated with the protein having the amino acid sequence SEQ ID no. 1.

By “molecular marker associated with the protein” it is meant that the molecular marker can be constituted of the protein itself or of any fragment of the latter, especially any fragment of a size higher or equal to 30 amino acids, or else by any nucleic acid associated with this protein, especially any gene segment coding this protein, or any segment situated upstream or downstream this gene and including the promoter and/or regulatory sequences of this gene.

In particular, the choice of a molecular marker of the nucleotide type proves to be entirely advantageous, as the analysis method according to the invention then makes it possible to perform a particularly reliable evaluation of the capacity of resistance to sulphites of the yeasts of the Brettanomyces bruxellensis species contained in the sample, by performing a genetic analysis of these yeasts.

The molecular marker, specific to yeasts of the Brettanomyces bruxellensis species of the oenological triploid yeasts group, can in particular be constituted of any segment of at least 90 successive nucleotides of the nucleotide sequence of sequence SEQ ID no. 2 or of any nucleotide sequence having with this sequence SEQ ID no. 2 a degree of homology higher or equal to 90% on 80% or more of the sequence. The nucleotide sequence SEQ ID no. 2 includes the gene coding the protein “putative histone acetyltransferase saga complex component” mentioned above, as well as the sequences containing the promoter and regulatory sequences of this gene, situated upstream and downstream with respect to the latter, of a size of about 1 Kb each.

Thus, in particular implementation modes of the invention, the detection step of the analysis method comprises the detection, in particular in an extract of nucleic acids obtained from yeasts contained in the sample, of a segment of at least 90 successive nucleotides of the nucleotide sequence of sequence SEQ ID no. 2 or of any nucleotide sequence having with this sequence, SEQ ID no. 2, a degree of homology higher or equal to 90% on 80% or more of the sequence.

Any conventional technique for the detection of a molecular marker can be implemented in the context of the invention.

When the molecular marker targeted by the detection step of the analysis method according to the invention is a peptide or a protein, this detection step can, for example, implement the technique known as Enzyme-linked Immunosorbent Assay ELISA.

When this molecular marker is a nucleotide sequence, the detection step of the analysis method according to the invention can, in particular, implement the technique of polymerase chain reaction (PCR).

Thus, in particular implementation modes of the invention, the detection step comprises:
the extraction of nucleic acids from yeasts contained in the sample,
the amplification, by polymerase chain reaction, of a locus containing the molecular marker specific to yeasts of the Brettanomyces bruxellensis species of the targeted oenological triploid yeasts group, and
the detection of said molecular marker in the DNA thus amplified, for example by separation of the amplicons through electrophoresis, especially in agarose gel or capillary, and size analysis.

As an example, the polymerase chain reaction can implement at least one nucleotide primer whose nucleotide sequence contains a sequence chosen among the sequences SEQ ID no. 3 and SEQ ID no. 4. It implements, preferably, a pair of nucleotide primers whose nucleotide sequences contain sequence SEQ ID no. 3 and sequence SEQ ID no. 4, respectively. When the implemented pair of nucleotide primers has the respective sequences SEQ ID no. 3 and SEQ ID no. 4, it allows amplifying the nucleotide sequence of sequence SEQ ID No. 7 of 263 pairs of bases.

The analysis method of the invention can be implemented starting from nucleic acids extracted from a mixture of yeasts contained in the sample, as well as from individual colonies of yeasts isolated from this sample.

Thus, in particular implementation modes of the invention, the detection step comprises:
the isolation, from a sample, of colonies of yeasts of the Brettanomyces bruxellensis species; this can, for example, be performed by spreading and culturing yeasts on a medium that is selective for the Brettanomyces bruxellensis species, for example on a medium called NS, for Non-Saccharomyces; such environments are well known by the person skilled in the art;
the extraction of nucleic acids, in particular of genomic DNA, from each of these colonies, and
the detection, in each of the nucleic acid extracts thus obtained, of said molecular marker.

This detection can in particular use the polymerase chain reaction technique, as explained above.

Such a mode of implementing the invention proves to be particularly advantageous, especially as it allows, easily and quickly, to count the colonies for which said molecular marker can be detected, in relation to the total number of analyzed colonies, and to deduce therefrom the approximate percent of yeasts present in the sample which are resistant to sulphites. Knowing this approximate percent proves in particular to be useful for the persons responsible with making the wines, for taking their decision whether to proceed to a sulphiting step during the wine-making process. Indeed, in cases where the yeasts of the Brettanomyces bruxellensis species which are resistant to sulphites are present in major amounts in the sample, whose yeasts contents is representative for that of the wine, it can be decided not to use sulphiting, which shall have little efficiency and which, in addition, would be associated with the risk of reinforcement of the resistance to sulphites of the species.

In particular implementation modes of the invention, the analysis method comprises, in addition, a step of verifying that the yeasts, for which the detection of the molecular marker specific to yeasts of the Brettanomyces bruxellensis species of the oenological triploid yeasts group is performed, belong indeed to the Brettanomyces bruxellensis species. This step can be performed beforehand or simultaneously with the detection step of the possible presence in the sample of yeasts of the Brettanomyces bruxellensis species belonging to the oenological triploid yeasts group. It can in particular be implemented by detection of a nucleotide sequence specific to the Brettanomyces bruxellensis species, for example, by amplification by means of polymerase chain reaction, with adequate nucleotide primers, and analysis of the size of the amplicons obtained, after separation by using, for example, electrophoresis.

As an example, the polymerase chain reaction, used for verifying if the yeasts belong to the Brettanomyces bruxellensis species, can implement at least one nucleotide primer whose nucleotide sequence contains a sequence chosen among the sequences SEQ ID no. 5 and SEQ ID no. 6. It implements, preferably, a pair of nucleotide primers of nucleotide sequences containing sequence SEQ ID no. 5 and sequence SEQ ID no. 6, respectively. The pair of primers of respective sequences SEQ ID no. 5 and SEQ ID no. 6, which is described in the publication of Ibeas et al., 1996, in Applied and Environmental Microbiology, 62: 998-1003, as specific to the Brettanomyces bruxellensis species, allows amplifying for this species a nucleotide sequence of 470 base pairs.

As an example, the analysis method according to the invention can comprise the implementation of a polymerase chain reaction, on genomic DNA extracted from yeasts contained in the sample, by means of two pairs of nucleotide primers, among which a pair of primers specific to the yeasts of the Brettanomyces bruxellensis species, and a pair of nucleotide primers specific to the yeasts of this species belonging to the genetic group of oenological triploid yeasts.

The first pair of nucleotide primers can in particular be the following:

Primer Db1:
(SEQ ID no. 5)
5′ AGAAGTTGAACGGCCGCATTTGCAC 3′
Primer Db2:
(SEQ ID no. 6)
5′ AGGATTGTTGACACTCTCGCCGAGG 3′

The second pair of nucleotide primers can in particular be the following:

Primer F1:
(SEQ ID no. 3)
5′ TCTTCCTCCGATCCTTTATCA 3′
Primer R1:
(SEQ ID no. 4)
5′ TTGCACGATTTGTCAGAATCC 3′

When these two pairs of nucleotide primers are implemented simultaneously, after separation of the amplicons by using electrophoresis, for example, in agarose gel or capillary, the method according to the invention comprises the analysis of the size of the fragments obtained. The amplification of a band at about 470 base pairs (bp) confirms the belonging to the Brettanomyces bruxellensis species. The amplification of this sole band at 470 bp indicates, in addition, the belonging to the genetic group of diploid yeasts; the amplification of an additional band at 356 bp indicates the belonging to the genetic group of beer triploid yeasts; and the amplification of an additional band at 263 bp indicates the belonging to the genetic group of oenological triploid yeasts.

The method of the invention thus advantageously makes it possible, in a single analysis and quickly, and, in addition, with equipment currently used in the oenological analysis laboratories, such as a thermocycler and an electrophoresis device, to establish the capacity of resistance to sulphites of yeasts of the Brettanomyces bruxellensis species contained in the sample and, in particular, of the majority population of such yeasts.

When the detection step and/or the verification step of the analysis method according to the invention make use of the polymerase chain reaction technique, this technique is implemented in a way that is conventional for the person skilled in the art. Thus, it can be performed, for example, starting from 50 to 500 ng of DNA, with the help of 1.5 to 3 μM of each nucleotide primer.

In the particular implementation modes of the invention, the detection step of the analysis method comprises a determination step for determining the quantity of yeasts of the Brettanomyces bruxellensis species belonging to the genetic group of oenological triploid yeasts present in the analyzed sample. This determination can be realized by any classic method known by the person skilled in the art, especially by quantitative polymerase chain reaction (qPCR), by means of a pair of primers capable of amplifying the molecular marker specific to the yeasts of the Brettanomyces bruxellensis species of the genetic group of oenological triploid yeasts, for example a pair of nucleotide primers of sequences containing sequence SEQ ID no. 3 and sequence SEQ ID no. 4, respectively.

The analysis method according to the invention can, in addition, comprise a step of determining the total quantity of yeasts of the Brettanomyces bruxellensis species present in the analyzed sample, in particular also by using qPCR.

In such implementation modes, the method according to the invention makes it advantageously possible to quantify exactly, on the one hand, the total population of yeasts of the Brettanomyces bruxellensis species present in the sample, and, on the other hand, the percent of yeasts of the Brettanomyces bruxellensis species that are resistant to sulphites.

According to another aspect, the invention concerns the use, for the analysis of the possible presence, in a sample, of sulphite-resistant yeasts of the Brettanomyces bruxellensis species, of a pair of nucleotide primers capable of amplifying, by polymerase chain reaction, a nucleotide sequence specific to the yeasts of the Brettanomyces bruxellensis species belonging to the genetic group of oenological triploid yeasts.

This pair of nucleotide primers can, in particular, be capable of amplifying a segment of at least 90 successive nucleotides of the nucleotide sequence of sequence SEQ ID no. 2 or of any nucleotide sequence having with this sequence SEQ ID no. 2 a degree of homology higher or equal to 90% on 80% or more of the sequence.

This pair of nucleotide primers can in particular be the following:

Primer F1:
(SEQ ID no. 3)
5′ TCTTCCTCCGATCCTTTATCA 3′
Primer R1:
(SEQ ID no. 4)
5′ TTGCACGATTTGTCAGAATCC 3′

or consist of a pair of primers of nucleotide sequences containing the sequence SEQ ID no. 3 and SEQ ID no. 4, respectively.

Another aspect of the invention concerns a kit for implementing the analysis method according to the invention, having one or several of the aforementioned characteristics. This kit comprises:
a pair of nucleotide primers for the amplification, by means of polymerase chain reaction, of a nucleotide sequence specific to the yeasts of the Brettanomyces bruxellensis species belonging to the genetic group of oenological triploid yeasts; and
a pair of nucleotide primers for the amplification, by means of polymerase chain reaction, of a nucleotide sequence evidencing the belonging of a yeast to the Brettanomyces bruxellensis species, specific to this species.

The kit presented in the invention can include in particular:
a pair of nucleotide primers for the amplification, by means of polymerase chain reaction, of a segment of at least 90 successive nucleotides of the nucleotide sequence of sequence SEQ ID no. 2 or of any other nucleotide sequence having with this sequence SEQ ID no. 2 a degree of homology higher or equal to 90% on 80% or more of the sequence, for example, a pair of nucleotide primers of nucleotide sequences containing sequence SEQ ID no. 3 and sequence SEQ ID no. 4, respectively; and/or
a pair of nucleotide primers of nucleotide sequences containing sequence SEQ ID no. 5 and sequence SEQ ID no. 6, respectively.

The kit according to the invention can as well include at least one, and in particular several, and, for example, all, of the following components:
a reagent for the polymerase chain reaction, such as Taq polymerase, optionally associated to an adapted buffer,
a reagent for qPCR, such as a SYBR® Green reagent or a nucleic probe marked for the TAQMAN® system,
a nucleic acids molecular size marker, in particular for analysis by electrophoresis,
a reagent for extracting nucleic acids from yeasts, and/or
instructions for implementing the steps of a method according to the invention.

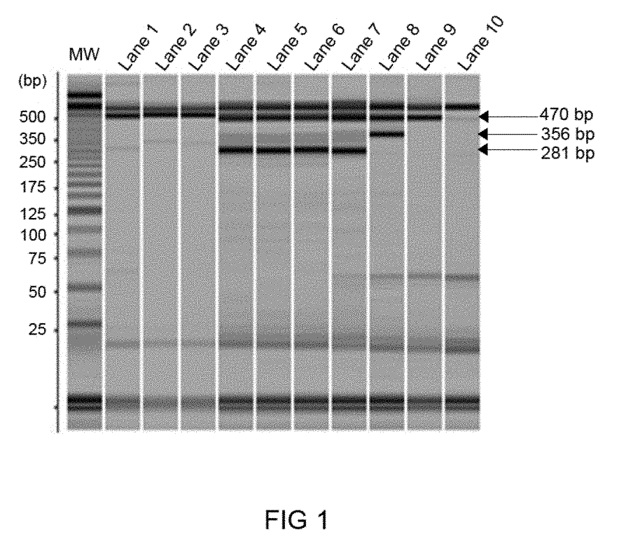

The characteristics and the advantages of the method of the invention shall be clearer when viewing the implementation examples presented below, supplied by way of illustration and not as a limitation of the invention, with the support of FIG. 1 that represents an electrophoresis gel made on the products of amplification using PCR, according to a protocol compliant with the invention, for: lanes 1 to 6, clones of yeasts of the Brettanomyces bruxellensis species, obtained from a sample of red wine matured in barrels; lane 7, a control sample known for its belonging to the genetic group of oenological triploid yeasts; lane 8, a control sample known for its belonging to the genetic group of beer triploid yeasts; lane 9, a control sample known for its belonging to the genetic group of oenological diploid yeasts; lane 10, a negative control; the first lane corresponds to the molecular size marker (MW).

### Example 1

The following method is implemented for determining, for 33 different strains, their distribution in the three genetic groups: oenological diploid, oenological triploid and beer triploid.

For each of these 33 strains, cells are collected from a spread of an a Petri dish, diluted individually in a solution of NaOH 20 mM to a concentration of about 10⁸ cells/mL, then the solution obtained is heated for 10 min at 94° C. This matrix, that contains the DNA, is used for the subsequent step of amplification using PCR.

The amplification step using PCR is performed according to the invention, using the following mixture:
3 μM of primer M13-F1 (SEQ ID no. 8: ACGACGTTGTAAAACGACTCTTCCTCCGATCCTTTATCA)
3 μM of primer R1 (SEQ ID No: 4: TTGCACGATTTGTCAGAATCC)
1.5 μM of primer Db1 (SEQ ID no. 5: AGAAGTTGAACGGCCGCATTTGCAC)
1.5 μM of primer Db2 (SEQ ID no. 6: AGGATTGTTGACACTCTCGCGGAGG)
Taq polymerase+adapted buffer (1× Taq-&GO® of MP Biomedicals)
1 μl of DNA matrix in NaOH solution (containing between 50 and 500 ng of DNA).

The primer M13-F1 includes the sequence SEQ ID no. 3, as well as, at its extremity 5′, the sequence of a tail M13 of 18 nucleotides.

The PCR program is the following:
5 min at 95° C.,
35 cycles of (30 s at 95° C.; 30 s at 57° C.; 1 min at 72° C.),
10 min at 72° C.

At the end of the amplification step using PCR, the amplicons are separated by electrophoresis in agarose gel 2%.

As a control, a PCR reaction is also performed using only the pair of primers Db1/Db2. The occurrence on the agarose gel of only one band at about 470 bp, for all the tested strains, confirms that these strains belong all to the Brettanomyces bruxellensis species.

The different strains of Brettanomyces bruxellensis are classified in three groups: the strains for which, after applying PCR with the two pairs of primers indicated above, only a band of about 470 bp appears, are classified as belonging to the oenological diploid yeasts group; the strains for which, in addition to this band at about 470 bp, an additional band at 356 bp appears, are classified as belonging to the beer triploid yeasts group; and the strains for which, in addition to the band at about 470 bp, an additional band at 281 bp appears, are classified as belonging to the oenological triploid yeasts group.

The 33 strains tested are thus distributed in: 17 strains of the genetic group of oenological diploid yeasts, 8 strains of the genetic group of oenological triploid yeasts and 8 strains of the genetic group of beer triploid yeasts.

The correlation between the triploidy of Brettanomyces bruxellensis strains and their sensitivity/resistance to sulphites is confirmed as follows.

The 33 strains of Brettanomyces bruxellensis are cultivated in a medium in the absence or the presence of different concentrations of sulphur dioxide SO₂ in the culture medium (0 mg/L, 0.2 mg/L, 0.4 mg/L and 0.6 mg/L).

After 14 days of culture, the capacity of the strains to grow in the different media is evaluated. The results obtained are indicated in the Table 1 below.

TABLE 1
Evaluation of the capacity of strains of each genetic
group to grow in the presence of sulphur dioxide

Number of strains with
the capacity to grow

| | | | | |
|---|---|---|---|---|
| Concentration in active SO₂ in the | 0 | 0.2 | 0.4 | 0.6 |
| culture medium (mg/L) | | | | |
| Oenological diploid (17 strains) | 17 | 6 | 1 | 0 |
| Beer triploid (8 strains) | 8 | 3 | 0 | 0 |
| Oenological triploid (8 strains) | 8 | 8 | 8 | 4 |

The relation between the genetic group and the capacity of resistance to sulphites is confirmed for all the tested strains.

### Example 2

A method according to the invention is implemented on a sample of a great wine of Châteaux X still aging.

The collection of this sample was made in October 2014 from a barrel.

Several quantities of this sample (10 ml and 100 μl) are spread on a selective medium called NS (for “Non-Saccharomyces”), containing: yeast extract (10 g/l), BactoPeptone (10 g/l), glucose (20 g/l), agar (20 g/l), chloramphenicol (0.1 mg/ml), biphenyl (0.15 mg/ml), actidione (0.5 mg/ml).

After 7 days of incubation at 30° C., it is observed, for the “10 ml” sample, the presence of a cell layer, and for the “100 μl” sample, isolated clones.

Six of these isolated clones are collected, diluted individually in a NaOH 20 mM solution, at a concentration of about 10⁸ cells/mL, then the solution is heated during 10 min at 94° C.

Each DNA matrix thus obtained is subjected to an amplification step using PCR, according to the operating conditions described in Example 1 above.

At the end of this step, each solution obtained is diluted at 1/2, and analyzed by capillary electrophoresis (MultiNA, Shimadzu).

Control strains known for their belonging to a given genetic group are subjected to the same method: oenological triploid control, beer triploid control, oenological diploid control, as well as a negative control where the DNA matrix is replaced with distilled water.

The electrophoresis gel obtained is shown in FIG. 1.

As it can be observed, out of the 6 clones tested, 3 clones belong to the oenological diploid group (sensitive to sulphites) (lanes 1 to 3) and 3 clones belong to the oenological triploid group (resistant to sulphites) (lanes 4 to 6). 50% of B. bruxellensis strains are thus resistant to sulphites, and 50% are sensitive to sulphites.

### Example 3—Quantitative Test

A method according to the invention is implemented on a sample of wine of Example 2.

An extraction of the total DNA of the sample is, first of all, performed by means of the commercial kit VINEO® Extract DNA Kit of Biorad.

The DNA matrix thus obtained is subjected to quantitative PCR. For this purpose, the following mixture is made:
1 to 10 μM of each of the primers M13-F1 and R1, and, optionally, of primers Db1 and Db2,
1× Taq polymerase+adapted buffer+SYBR® Green PCR Supermix of Bio-Rad,
50-100 ng of DNA extract.

A standard range is also performed from a sample with known concentration in one strain of Brettanomyces bruxellensis known as belonging to the oenological triploid group.

The quantity of yeasts of the Brettanomyces bruxellensis species belonging to the oenological triploid group, thus resistant to sulphites, present in the initial wine sample, is established.

In parallel, the total quantity of yeasts of the Brettanomyces bruxellensis species present in the wine sample can also be determined, for example, in a way that is classic per se, by means of the kit VINEO® Brettanomytest Kit PCR of the Biorad Company.

1-15. (canceled)

16. An analysis method for analyzing a sample for a presence of sulphite-resistant yeasts of Brettanomyces bruxellensis species, comprising the steps of:
detecting a presence of yeasts of the Brettanomyces bruxellensis species belonging to a genetic group of oenological triploid yeasts in said sample, and
deducing said sample contains the yeasts of the Brettanomyces bruxellensis species resistant to sulphites in response to the presence detected in said sample.

17. The analysis method according to claim 16, wherein the step of detecting the presence further comprises a step of detecting, from yeasts contained in the sample, a molecular marker specific to the yeasts of the Brettanomyces bruxellensis species of the genetic group of oenological triploid yeasts.

18. The analysis method according to claim 17, wherein said molecular marker is associated with a protein having an amino acid sequence SEQ ID no. 1.

19. The analysis method according to claim 17, wherein said molecular marker is constituted of any segment of at least 90 successive nucleotides of a nucleotide sequence SEQ ID no. 2.

20. The analysis method according to claim 17, wherein the step of detecting the presence further comprises steps of:
extracting nucleic acids from the yeasts contained in said sample;
amplifying a locus containing said molecular marker by a polymerase chain reaction; and
detecting said molecular marker in an amplified DNA.

21. The analysis method according to claim 20, wherein the polymerase chain reaction implements at least a nucleotide primer, a sequence of which contains a sequence selected from the group consisting of sequences SEQ ID no. 3 and SEQ ID no 4.

22. The analysis method according to claim 17, wherein the step of detecting the presence further comprises steps of:
isolating colonies of the yeasts of the Brettanomyces bruxellensis species from said sample;
extracting nucleic acids from each of the colonies; and
detecting said molecular marker in each of nucleic acid extracts obtained.

23. The analysis method according to claim 17, further comprising a step of verifying that the yeasts, upon which said molecular marker detection was performed, belong to the Brettanomyces bruxellensis species.

24. The analysis method according to claim 16, wherein the step of detecting the presence further comprises a step of determining a quantity of the yeasts of the Brettanomyces bruxellensis species belonging to the genetic group of oenological triploid yeasts present in said sample.

25. The analysis method according to claim 24, wherein the step of determining is realized by a quantitative polymerase chain reaction.

26. The analysis method according to claim 16, further comprising a step of determining a quantity of yeasts of the Brettanomyces bruxellensis species present in said sample.

27. The analysis method according to claim 17, wherein said molecular marker is constituted of any segment of at least 90 successive nucleotides of a nucleotide sequence SEQ ID no. 2 having a degree of homology higher than or equal to 90% on 80% or more of the nucleotide sequence.

28. A method of using a pair of nucleotide primers capable of amplifying, by a polymerase chain reaction, a nucleotide sequence specific to yeasts of the Brettanomyces bruxellensis species belonging to a genetic group of oenological triploid yeasts, to analyze a sample for a presence of sulphite-resistant yeasts of the Brettanomyces bruxellensis species.

29. The method according to claim 28, wherein said pair of nucleotide primers is capable of amplifying a segment of at least 90 successive nucleotides of a nucleotide sequence SEQ ID no. 2.

30. The method according to claim 28, wherein said pair of nucleotide primers is capable of amplifying a segment of at least 90 successive nucleotides of a nucleotide sequence SEQ ID no. 2 having a degree of homology higher than or equal to 90% on 80% or more of the nucleotide sequence.

31. A kit for implementing an analysis method according to claim 16, comprising:
a pair of nucleotide primers to amplify, by a polymerase chain reaction, a nucleotide sequence specific to yeasts of the Brettanomyces bruxellensis species belonging to a genetic group of oenological triploid yeasts; and
a pair of nucleotide primers to amplify, by the polymerase chain reaction, a nucleotide sequence of a yeast evidencing belonging to the Brettanomyces bruxellensis species.

32. The kit according to claim 31, further comprising a pair of nucleotide primers to amplify, by the polymerase chain reaction, a segment of at least 90 successive nucleotides of a nucleotide sequence SEQ ID no. 2.

33. The kit according to claim 31, further comprising a pair of nucleotide primers to amplify, by the polymerase chain reaction, a segment of at least 90 successive nucleotides of a nucleotide sequence SEQ ID no. 2 having a degree of homology higher than or equal to 90% on 80% or more of the nucleotide sequence.

34. The Kit according to claim 31, further comprising a pair of nucleotide primers of sequences containing a sequence SEQ ID no. 5 and a sequence SEQ ID no. 6, respectively.
